(19) United States
(12) Patent Application Publication
(10) Pub. No.: US 2009/0143238 A1
(43) Pub. Date: Jun. 4, 2009

(54) Oligonucleotide matrix and methods of use

(76) Inventors: Junghuei Chen, Newark, DE (US); Yuzhen Wang, Newark, DE (US); Yu-Chang Tsai, Newark, DE (US)

Correspondence Address: MCCARTER & ENGLISH, LLP WILMINGTON;BASIL S. KRIKELIS, Renaissance Centre, 405 N. King Street, 8th Floor, WILMINGTON, DE, 19801, US

(21) Appl. No.: 11/656,244

(22) Filed: Jan. 22, 2007

Related U.S. Application Data
(60) Provisional application No. 60/761,308, filed on Jan. 23, 2006.

(51) Int. Cl. C40B 30/04; C07H 21/04; C07H 21/02; C40B 40/08; C40B 50/06

(52) U.S. Cl. 506/9; 536/24.33; 506/17; 506/26

(57) ABSTRACT

The present invention relates broadly to compositions and methods for performing nucleic acid analysis. In particular the invention relates to a universal oligonucleotide probe set and a hybridization matrix or array for performing analysis of nucleic acids from any source. The oligonucleotide matrix of the present invention provides up to approximately 10¹⁸ different oligonucleotides thus being sensitive enough to provide unprecedented capacity and sensitivity.

### CROSS REFERENCE TO RELATED APPLICATIONS

The present application claims the benefit of U.S. Provisional Application Ser. No. filed 60/761,308, Filed Jan. 23, 2006, the disclosure of which is expressly incorporated herein by reference in its entirety.

### RELATED FEDERALLY SPONSORED RESEARCH

The work described in this application was sponsored by the National Science Foundation (NSF) under Contract Numbers MCB-9980092 and EIA-0130385.

### SEQUENCE LISTING

This application explicitly includes the nucleotide sequences numbers: 1-24, which are also provided in the Sequence Listing contained on the disc labeled with the following: Docket No. 99689-00044; Applicant: Junghuei Chen, et al.; Title: Oligonucleotide Matrix and Methods of Use; Format: ASCII; SEQUENCE LISTING, Date Created: Jan. 22, 2007; Size: 5 kb; which is submitted herewith, and hereby incorporated by reference in its entirety.

### FIELD OF THE INVENTION

The present invention relates broadly to compositions and methods for performing nucleic acid analysis of an organism. In particular the invention relates to the production of an oligonucleotide probe set and method of use in a matrix for performing analysis of genetic material regardless of its biological source.

### BACKGROUND OF THE INVENTION

It is axiomatic that variations in gene expression can be used as a reliable indicator of the phenotypic state of an organism. By comparing gene expression in closely related cells which exhibit distinct phenotypes, for example, malignant and nonmalignant cells from the same tissue origin, correlations may be drawn between gene expression and phenotype. A better understanding of the temporal and spatial gene expression relationships in health and disease will increase understanding of the disease states and may lead to identification of new drug targets or other therapeutic interventions for a variety of diseases.

Scientists have the ability to assess the changes in expression of multiple genes in cells of different types or at different stages of differentiation or cell cycle. Efforts to correlate gene expression alterations with a particular phenotype have been greatly aided by the sequencing of the genomes of various organisms and identification of all genes encoded therein. For example, the emerging field of biomarkers is focused on the search for discrete genes, and/or their cognate proteins that serve as high confidence indicators of specific disease states. However, the diagnostic utility of biomarkers requires that a clinician correctly determine what disease, and therefore, which biomarkers should be assayed, and/or requires that tests for all currently known biomarkers be performed.

As suggested above, current observation and measurement methods suffer from one or more disadvantages that render them narrow in scope, unnecessarily inaccurate, time consuming, labor intensive, or expensive. Such disadvantages flow from requirements, such as the prior knowledge of gene sequences, limited transcript coverage, inability to detect sequence variants, limited application to a particular species, repetitive sequencing of sample nucleic acids, and so forth. Currently, there is no platform available that allows for the efficient and economic profiling of entire genomes, and/or transcriptomes but which can also be used with any species of interest.

Therefore, any observational method that can rapidly, accurately, and economically observe and measure the presence or expression of whole genomes or of selected individual genes will be of great value. Of even more value will be methods that can directly and quantitatively be applied to the complex mixtures of DNA, for example, genomic DNA (“gDNA”) samples or expressed DNA (“cDNA”) samples (synthesized from selected RNA pools) that are typically derived directly from biological samples.

### SUMMARY OF THE INVENTION

The invention relates broadly to compositions and methods for observing and measuring the presence and expression of genes, entire genomes or transcriptomes (i.e., the subpopulation of genes which are expressed by a cell at any given time under a given physiological state), regardless of the species of interest. In particular, the compositions and methods of the invention make accurate and efficient use of arrayed oligonucleotides to avoid any requirements for cloning of complex mixtures of sequences into many individual samples of a single sequence, repetitive sequencing of sample components, and so forth.

Additional objects and advantages of the present invention will be appreciated by one of ordinary skill in the art in light of the current description and examples of the preferred embodiments, and are expressly included within the scope of the present invention.

### BRIEF DESCRIPTION OF THE DRAWINGS

FIG. 1 is a diagram showing how the oligonucleotide matrix can be transferred to a hybridization support membrane and used as a universal probe for any labeled input nucleic acid; a detector monitors the labeled input nucleic acid that remains bound to the membrane and the data is analyzed by computer to provide a “signature” for the input source.

FIG. 2 is a schematic demonstrating the construction of the universal oligonucleotide library step of the invention. First, oligonucleotides comprising a random segment and a primer segment are synthesized; the oligos are amplified using PCR primer-based extension with a primer complementary to said primer segment

FIG. 3 is a schematic demonstrating the electrophoresis separation step of the library of double-stranded universal oligonucleotides; the oligos are separated into single-stranded nucleic acids on a 1-D denaturing gradient gel according to their GC content.

FIG. 4 shows a gel electrophoresis separation of a number of double-stranded oligonucleotides (A) according to the method of the invention. The results (B) indicate that the electrophoresis step accurately and predictably separates the oligos according to their GC content (i.e., Tₘ).

FIG. 5 shows another gel electrophoresis separation of a number of double-stranded oligonucleotides according to the method of the invention. In this gel, however, the double-stranded oligonucleotides were mixed prior to separation on the gel. This result shows that complex mixtures of double-stranded oligonucleotides can be reliably separated on the denaturing gel into discrete bands according to their melting temperatures. Lane 1 contains a mixture of 4 duplexes, lane 2 contains another 4 duplexes, lane 3 contains a mixture of lanes 1 and 2 (i.e., 8 total duplexes). Lane 4 contains a mixture of another 7 duplexes, and lane 5 is a mixture of lanes 3 and 4 (i.e., 15 total duplexes).

FIG. 6 shows the separation of DNA oligonucleotide libraries or sets in the gradient denaturing gel system. (A) DNA oligonucleotide libraries contain increasing numbers of difference sequences; from 2⁴ (4M) to 2²⁰ (20M). (B) Another set of libraries contain 2¹² (12M) to 2²⁰ (20M) different sequences.

FIG. 7 depicts an aspect of the oligonucleotide matrix of the invention. In this aspect the denatured and electrophoresed oligonucleotide set is further separated in a second dimension (i.e., a two-dimensional matrix) in a non-denaturing gel electrophoresis step. In this process the oligonucleotides are separated by their secondary structures in a non-denaturing gel. Molecules running at the same position in the first dimension (denaturing) would have the same melting temperature but will form different secondary structure under native conditions of the second ge. The 2-D oligo matrix is then transferred to a hybridization support membrane and used as a universal probe set or library for any labeled input nucleic acid; a detector monitors the labeled input nucleic acid that remains bound to the membrane and the data is analyzed by computer to provide a “signature” for the input source.

FIG. 8 depicts a second aspect of the universal oligonucleotide matrix of the invention. In this aspect multiple oligonucleotide sets or libraries, representing multiple synthesis reactions, are separated in the denaturing electrophoresis gel. The separated oligonucleotide sets from multiple denaturing electrophoresis runs are excised from the gel, aligned in parallel and transferred to a hybridization support membrane and used as a universal oligonucleotide probe set or library for any labeled input nucleic acid; a detector monitors the labeled input nucleic acid that remains bound to the membrane and the data is analyzed by computer to provide a “signature” for the input source.

FIG. 9 is an example of a universal oligonucleotide matrix of the invention with 8 million different 40-mers probed with three different radioactive labeled DNA (A, theta chi phage; B, lambda phage; C, random 20-mer DNA).

### DETAILED DESCRIPTION OF THE INVENTION

The present invention relates to the surprising discovery that a universal oligonucleotide matrix can be generated that contains sufficient sequence diversity to allow signal profiling for entire genomes or transcriptomes, regardless of the species of interest. A further advantage to the present invention is that the genome-wide profiling method is highly reproducible, and can be performed economically and efficiently.

The universal oligonucleotide matrix of the invention is sensitive enough to provide unprecedented capacity and sensitivity as it is not limited to chip size. The effectiveness of the system is based, in theory, on the statistical likelihood that there will be at least one random polynucleotide segment of length n in the matrix that is complementary, and therefore, capable of undergoing specific hybridization with at least a portion of every and any input nucleic acid.

In certain aspects the universal oligonucleotide matrix of the present invention comprises a large set or library of synthesized oligonucleotides with up to approximately 10¹⁸ different, randomly generated 30- or 40-mers in a matrix that is capable of monitoring or profiling the existence of any input biological or non-biological DNA or RNA samples. The random polynucleotide segment of the oligonucleotides in the matrix of the invention undergo specific Watson-Crick base pairing or hybridization with a complementary sequence or subsequence of the labeled input nucleic acid. In any of the embodiments described herein, the oligonucleotides of the matrix can be synthesized from deoxyribonucleotides, ribonucleotides, modified nucleotides, nucleotide analogs, for example, PNAs, or any combination thereof.

In one aspect the present invention relates to compositions for performing nucleic acid analysis. This aspect of the invention includes the use of an oligonucleotide set, wherein each oligonucleotide of the set comprises a random polynucleotide segment (“random segment”) of from about 15 to about 50 nucleotides which is covalently linked or contiguous with at least one primer polynucleotide segment (“primer segment”) of from about 10 to about 40 nucleotides. In a preferred embodiment, the primer segment is a known nucleotide sequence. Preferably, the random polynucleotide segments are generated by a conventional oligonucleotide synthesizer (e.g., those produced by ABI®, Thuramed®, Beckman®, or Qiagen®) under conditions where each nucleotide species, i.e., A, C, T (or U), and G, is present at equal molar concentrations such that at each discrete position along the 20-40mer, each nucleotide has an equal probability of being present. The random segment can be used in a subsequent oligonucleotide synthesis reaction where the addition of nucleotides is controlled such that a specific primer polynucleotide sequence results, contiguous with the random segment. In another embodiment the random segment is from about 25 to about 35 nucleotides long. In still another embodiment, the primer segment is from about 15 to about 25 nucleotides long.

In a related aspect, the present invention includes a population or set of oligonucleotides or polynucleotides useful for performing nucleic acid analysis, and methods for their synthesis. The oligonucleotide set comprises approximately 4ⁿ individual oligonucleotides, wherein n represents the number of nucleotides in the random segment and is an integer from 15 to 50, and wherein the random segment is covalently linked or contiguous with at least one known primer polynucleotide segment of from about 10 to about 40 nucleotides. For example, the oligonucleotide set generated using a random segment of 20 nucleotides comprises approximately 4²⁰ (or approximately 10¹²) unique polynucleotide sequences. Each individual random segment is then covalently linked to at least one known primer polynucleotide segment to generate the universal oligonucleotide set of the invention. In one embodiment, the oligonucleotide set comprises about 10¹⁸ individual oligonucleotides (i.e., a random segment 30 nucleotides in length covalently linked to at least one primer segment). In any of the preferred embodiments the primer segment can be adapted, for example, to hybridize with a complementary oligonucleotide to facilitate PCR extension and amplification, to contain unique recognition sites for restriction endonuclease (RE) enzymes, to contain labeled nucleotides, modified nucleotides, nucleotide analogs or the like. In certain aspects the oligonucleotides of the matrix are 50%, 60%, 70%, 80%, 90%, or 100% complementary to an input nucleic acid sequence such that hybridization occurs. Hybridization conditions can be of any degree of stringency, for example, low, medium, or high. In a preferred embodiment, a degree of stringency is selected such that non-specific binding of input nucleic acid to the matrix oligonucleotides is minimized resulting in a high signal to noise ratio.

In addition the invention relates to methods for generating a universal oligonucleotide set comprising the steps of synthesizing a population of random polynucleotide segments of length n by performing the oligonucleotide synthesis in the presence of equal molar concentrations of nucleotides A, C, T (or U), and G or derivatives thereof; and covalently linking substantially all random segments to at least one known primer polynucleotide segment of from about 10 to about 30 nucleotides in length, and wherein the number of oligonucleotides in the set is approximated by the formula, 4ⁿ, wherein n represents the number of nucleotides in the random probe segment and is an integer from 15 to 50. In one embodiment, the random segments are used in a subsequent oligonucleotide synthesis reaction where the nucleotide mixture is controlled resulting in a specific primer polynucleotide segment, contiguous with the random segment.

A related aspect of the invention includes compositions and methods for generating a universal oligonucleotide matrix. The method of the invention comprises the first step of generating a universal oligonucleotide set, wherein the oligonucleotides are comprised of a random polynucleotide segment of from 15 to 50 nucleotides as described above, and wherein each random segment is covalently linked or contiguous to at least one known primer segment of from 10 to 40 nucleotides. The oligonucleotide set is then used in a primer-based polymerase chain reaction (PCR) to amplify and generate double stranded universal oligonucleotide set.

To initiate the PCR reaction, an extension primer is added that is complementary to the primer segment of the oligonucleotide of the invention. After the PCR extension reaction is completed, the oligonucleotides are precipitated using standard techniques (e.g., chloroform extraction, and EtOH precipitation). The oligonucleotides are then subjected to electrophoresis separation in a denaturing gradient gel under suitable ionic and pH buffering conditions (e.g., pH 8.0, and TAE (Tris-acetate-EDTA) or TBE (Tris-borate-EDTA) buffer). The denaturing gel results in the separation of the double-stranded (ds) oligonucleotides based on the GC content of the oligo (i.e., is sequence/melting point dependent).

In certain aspects the denaturing gel comprises, a gradient of from about 0% to 100% of a mixture comprising from about 0.01M to about 9M urea, and from about 0% to about 60% formamide. In another embodiment the non-denaturing gel comprises a polyacrylamide gradient of from about 20% to about 10%. In any of the embodiments the hybridization support can be, for example, nitrocellulose, glass, DEAE dextran, PVDF, nylon, silicon, or other suitable nucleic acid binding material.

In one embodiment, the universal matrix comprises the separation of the oligonucleotide set in a second dimension. For example, the resulting denaturing gel-separated oligonucleotide set is subjected to a second electrophoresis separation on a non-denaturing gradient gel in a second dimension or direction that lies within the same plane but is rotated about 90 degrees relative to the first dimension. Single-stranded DNA mobility is sequence dependent because of varying degrees of intrastrand base pairing and the resulting looping and compaction.

The two electrophoresis steps result in a 2-D oligonucleotide matrix which is then transferred to a hybridization support under appropriate buffering conditions (See, Southern, E. M. 1975. J. Mol. Biol. 98:503; Sambrook, J., Fritsch, E. F., and Maniatis, T. 1989. Molecular Cloning, A Laboratory Manual, 2nd Ed. Cold Spring Harbor Laboratory Press, Cold Spring Harbor, N.Y.; which is incorporated herein by reference in its entirety and for all purposes).

In another embodiment, the universal oligonucleotide matrix comprises multiple denaturing gel-separated oligonucleotide libraries or sets spaced in parallel and transferred to a hybridization support under suitable conditions (See, See, Southern, E. M. 1975. J. Mol. Biol. 98:503; Sambrook, J., Fritsch, E. F., and Maniatis, T. 1989. Molecular Cloning, A Laboratory Manual, 2nd Ed. Cold Spring Harbor Laboratory Press, Cold Spring Harbor, N.Y.; which is incorporated herein by reference in its entirety and for all purposes). In certain aspects, this embodiment comprises the steps of isolating the products of multiple oligonucleotide synthesis reactions and separation on a denaturing gradient gel as described. The areas of the gel containing the separated and denatured oligonucleotide libraries can be excised and aligned in parallel for transfer to a hybridization support membrane. This approach results in the creation of a universal oligonucleotide matrix comprising multiple 1-D oligo sets in parallel (See Example #2).

Employing the universal oligonucleotide matrix of the invention, hybridization signatures of input or sample nucleic acids are produced. For example, when using human genomic DNA as an input, the resultant output from the matrix of the invention represents the genomic “signature” or “profile” of the subject. The matrix of the invention has a high enough sensitivity to detect minor differences of any closely related input nucleic acids. In other words, the present invention can generate genomic and gene expression signatures of specific organisms, including organ types, tissue types, and/or cell types that are indicative of different species, phenotypes, or diseases. Similarly, in the case of human genomic DNA for example, DNA of different races, gender, age and/or other traits generate distinctive signatures on the matrix of the invention.

The present invention also provides methods for performing nucleic acid analysis using the universal oligonucleotide matrix of the invention comprising providing an input nucleic acid, for example, RNA, or DNA isolated from the cell of an organism, suitably labeled with, for example, fluorescent or radioactive label, and incubating the labeled input nucleic acid with the support-bound universal oligonucleotide matrix under suitable hybridization conditions. The resulting signals from the input nucleic acid are then analyzed with a detector to generate a “signature” or “profile” for the specific input nucleic acid source. The input nucleic acid samples can be, for example, RNA or DNA (“DNA” refers to cDNA or gDNA, however derived or synthesized, and includes nucleotide derivatives, mimetics, and analogs, for example, PNAs).

In another aspect the invention relates to a computer database comprising signatures or profiles generated using the universal oligonucleotide matrix and the methods of the invention. The database may contain profiles from a plurality of organisms, organ types, tissues types, and/or cellular types under a variety of physiological conditions. In a related aspect the invention includes a statistical based algorithm suitable for performing comparisons between a test or unknown signature and those contained in the database. For example, a computer algorithm could be used to analyze and compare regions of the reference signature and the test signature to determine the degree of statistical significance in identity of the reference to the test signatures.

The present invention also provides methods for detecting/identifying the presence of a particular nucleic acid source. In one aspect, the method comprises using the universal oligonucleotide matrix and the methods of the invention to generate a database of “reference signatures” based on input nucleic acid from many different biological sources, for example, virus, bacteria, fungi, yeast, unicellular organisms, plants, and animals. Next, nucleic acid is isolated from an unknown or unidentified source and suitably labeled. Using the methods of the invention, a “test signature” is generated using the nucleic acid from the unknown source. Lastly, the test signature is compared to the database of reference signatures to determine the identity of the organism. This method is particularly useful, for example, in the detection/identification of a potential pathogenic organism such as a virus or a bacteria.

In certain aspects the invention includes methods for determining exposure, infection, or contamination of an organism by a pathogenic organism. For example, the signature of the test subject can be compared to a database of host and pathogen signatures. Due to the reproducibility of the universal oligonucleotide matrix it is possible to determine the combination of genomes or transcriptomes present in a sample by comparing the test profile with combinations of known or reference profiles in the database.

In a related aspect the invention relates to methods for monitoring diseases, disease progression, and/or disease states in a subject. The methods of the invention involve using the oligonucleotide matrix and the methods of the invention to generate a database of “diagnostic signatures” comprising the nucleic acid signatures of many normal and disease tissues and disease states. Next, using the methods of the invention, a “test signature” is generated by isolating the nucleic acid of cell or tissue source whose pathological state is unknown Lastly, the test signature is compared to the database of diagnostic signatures to diagnose the particular disease or disease state. In a hypothetical example, the database comprises diagnostic signatures for normal prostate tissue, hyperplastic prostate, premalignant and malignant prostate tissue. Next, a test signature is generated using prostate tissue from a patient. A comparison of the patient's test signature with the diagnostic signature database would then allow a health care worker to determine which, if any, pathological state the patient exhibits. A diagnosis of this type is particularly useful because it provides the health care worker with more specific information regarding disease state/progression, leading to more efficient treatment courses, and improving prognoses and overall clinical outcomes.

The present invention also provides methods for monitoring the efficacy or response of therapy upon a subject (i.e., performing pharmacogenomic studies). The methods involve comparing a test or diagnostic signature, obtained from a subject undergoing a particular therapy, with a database of “response signatures” which are obtained from subjects whose response to a therapeutic is known. The detection and statistical analysis is performed as described above. From this comparison a health care worker can tailor a treatment plan for a particular subject that will promote the most optimum clinical outcome possible.

The methods of the invention are based at least in part on the discovery that perturbations on various constituents of a cell, such as, for example, perturbations on protein function or activity, which occur as a result of a disease state or therapy result in characteristic changes in the transcription and activity of other genes, and that such changes can be used to define a “signature” of the particular alterations which are correlated with the progression of the particular disease state or therapy. This is true even if there is no actual disruption in the function or activity level of proteins associated with the disease state. Further, the methods of the invention can be used to monitor several diseases and/or therapies simultaneously.

In still another aspect, the universal oligonucleotide matrix of the invention can be used to perform high-throughput screening of therapeutic molecules from a “library” of compounds of unknown biological utility or effect. For example, in this method a database of high-confidence drug “response signatures” could be generated using input nucleic acid from subjects treated with a known a class of therapeutics. For example, using a mouse model, a database of signatures could be generated from brain tissue in response to known anxiolytic drugs. Next, “test signatures,” can be generated from the brains of mice treated with a library of compounds. A comparison of the testy signature with response signatures for many known drugs and drug classes would allow a quick, cost effective, and efficient method for identifying potentially useful drugs, and the indication for which they might be most useful. This method of the invention, therefore, provides a means for dramatically reducing the time and cost of bringing novel therapeutics to market.

In still another aspect, the invention includes methods for identifying nucleic acid sequences that are associated with a particular physiological state and which are useful, for example, as biomarkers (i.e., genes whose presence, absence, or change is so closely correlated with a physiological state as to constitute, in essence, signposts of that physiological state), as therapeutics or as therapeutic targets. This method comprises isolating the region of interest on the universal oligonucleotide matrix that contains the labeled input nucleic acid, for example, by hole punch. Next, the nucleic acids binding is disrupted using a suitable denaturing buffer, for example, high ionic buffer, formamide, urea, high temperature or the like. Next, the nucleic acids are purified using techniques widely known in the art, and sequenced. The resulting sequence can be used both to generate primers for cloning of the desired transcript, and used to query a sequence database to determine its identity.

In yet another embodiment, the invention also provides kits comprising at least one container, the oligonucleotide set of the invention, and instructions for their use disposed therein.

### DEFINITIONS

“Sequence homology” refers to the proportion of base matches between two nucleic acid sequences or the proportion of amino acid matches between two amino acid sequences. When sequence homology is expressed as a percentage, for example 50%, the percentage denotes the proportion of matches of the length of sequences from a desired sequence that is compared to some other sequence. When using oligonucleotides as probes or treatments, the sequence homology between the target nucleic acid and the oligonucleotide sequence is generally not less than 30%.

The term “hybridization” refers to duplex formation between two or more polynucleotides, e.g., to form a double-stranded nucleic acid. The ability of two regions of complementarity to hybridize and remain together depends of the length and continuity of the complementary regions, and the stringency of hybridization conditions. In describing hybridization between any two nucleic acids (e.g., between an array probe and an amplified RNA target such as a cDNA), sometimes the hybridization encompasses “at least a portion” of the target or probe. As used herein, the phrase “at least a portion” and similar phrases in reference to hybridization reactions refer to a domain of complementarity that is sufficiently large to permit sequence-specific hybridization, e.g., allows stable duplex formation under stringent hybridization conditions.

“Selectively hybridize”, “selective hybridizing”, “hybridization” or “hybridizing” refers to at least two molecules that can detectably and specifically bind. For example, a molecule can be a polynucleotides, oligonucleotides and fragments thereof that selectively hybridize to target nucleic acid strands, under hybridization and wash conditions that minimize appreciable amounts of detectable binding to nonspecific nucleic acids. High stringency conditions can be used to achieve selective hybridization conditions as known in the art. Generally, the nucleic acid sequence homology between the polynucleotides, oligonucleotides, and fragments thereof and a nucleic acid sequence of interest will be at least 30%, and more typically and preferably of at least 40%, 50%, 60%, 70%, 80% or 90%. Hybridizations are usually performed under stringent conditions, for example, at a salt concentration of no more than 1M and a temperature of 25-30° C. For example, conditions of 5×SSPE (750 mM NaCl, 50 mM Na Phosphate, 5 mM EDTA, pH 7.4) and a temperature of 25-30° C. are suitable for allele-specific probe hybridizations.

The term “nucleotide” refers generally to a phosphate ester of a nucleoside, as a monomer unit or within a polynucleotide.

The terms “nucleic acid,” “nucleic acid sequence,” “polynucleotide,” “polynucleotide sequence,” “oligonucleotide,” “oligomer,” “oligo” or the like, as used herein, refer to a polymer of monomers subunits that can be corresponded to a sequence of cDNA, genomic DNA, synthesized nucleic acid, nuclear RNAs, peptide nucleic acid (PNA), RNA/DNA copolymers, any analogues thereof, or the like. A polynucleotide can be single- or double-stranded, and can be complementary to the sense or antisense strand of a gene sequence. A polynucleotide can hybridize with a complementary portion of a target polynucleotide to form a duplex, which can be a homoduplex or a heteroduplex. The length of a polynucleotide is not limited in any respect. Preferably, the oligonucleotides of the present invention range from 25-90 nucleotides. Oligonucleotides of the invention include sequences of deoxyribonucleic acid (DNA) or ribonucleic acid (RNA) or mimetics thereof which may be isolated from natural sources, recombinantly produced or artificially synthesized.

Linkages between nucleotides can be internucleotide-type phosphodiester linkages, or any other type of linkage. A “polynucleotide” refers to the sequence of nucleotide monomers along the polymer. A “polynucleotide” is not limited to any particular length or range of nucleotide sequence, as the term “polynucleotide” encompasses polymeric forms of nucleotides of any length. A polynucleotide can be produced by biological means (e.g., enzymatically), or synthesized using an enzyme-free system. A polynucleotide can be enzymatically extendable or enzymatically non-extendable. Unless otherwise indicated, a particular polynucleotide sequence of the invention optionally encompasses complementary sequences, in addition to the sequence explicitly indicated. Nucleic acid can be obtained from any source, for example, a cellular extract, genomic or extragenomic DNA, viral RNA or DNA, or artificially/chemically synthesized molecules.

Polynucleotides that are formed by 3′-5′phosphodiester linkages are said to have 5′-ends and 3′-ends because the nucleotide monomers that are reacted to make the polynucleotide are joined in such a manner that the 5′ phosphate of one mononucleotide pentose ring is attached to the 3′oxygen (hydroxyl) of its neighbor in one direction via the phosphodiester linkage. Thus, the 5′-end of a polynucleotide molecule has a free phosphate group or a hydroxyl at the 5′ position of the pentose ring of the nucleotide, while the 3′ end of the polynucleotide molecule has a free phosphate or hydroxyl group at the 3′ position of the pentose ring. Within a polynucleotide molecule, a position or sequence that is oriented 5′ relative to another position or sequence is said to be located “upstream,” while a position that is 3′ to another position is said to be “downstream.” This terminology reflects the fact that polymerases proceed and extend a polynucleotide chain in a 5′ to 3′ fashion along the template strand. Unless denoted otherwise, whenever a polynucleotide sequence is represented, it will be understood that the nucleotides are in 5′ to 3′ orientation from left to right.

As used herein, it is not intended that the term “polynucleotides” be limited to naturally occurring polynucleotides sequences or polynucleotide structures, naturally occurring backbones or naturally occurring internucleotide linkages. One familiar with the art knows well the wide variety of polynucleotide analogues, unnatural nucleotides, non-natural phosphodiester bond linkages and internucleotide analogs that find use with the invention. Non-limiting examples of such unnatural structures include non-ribose sugar backbones, 3′-5′ and 2′-5 phosphodiester linkages, pseudoisocytidine, 6-azacytidine, N⁶-methyl-dnTP, diaminopurine, purine, 2-aminopurine, and 2′-O-methyl-dnTP, 2′-deoxy-2′-fluoro-dnTP, internucleotide inverted linkages (e.g., 3′-3′ and 5′-5′), 7-deaza-dnTPs, branched structures, and internucleotide analogs (e.g., peptide nucleic acids (PNAs), locked nucleic acids (LNAs), alkylphosphonate linkages such as methylphosphonate, phosphoramidate, alkyl-phosphotriester, phosphorothioate and phosphorodithioate internucleotide linkages, and the like. Furthermore, a polynucleotide can be composed entirely of a single type of monomeric subunit and one type of linkage, or can be composed of mixtures or combinations of different types of subunits and different types of linkages (a polynucleotide can be a chimeric molecule). As used herein, a polynucleotide analog retains the essential nature of natural polynucleotides in that they hybridize to a single-stranded nucleic acid target in a manner similar to naturally occurring polynucleotides.

The term “RNA,” an acronym for ribonucleic acid, refers to any polymer of ribonucleotides. The term “RNA” can refer to polymers comprising natural, unnatural or modified ribonucleotides, or any combinations thereof (i.e., chimeric RNA molecules). The term “RNA” includes all biological forms of RNA, including for example, mRNA (typically polyA RNA), rRNA, tRNA, and small nuclear RNAs, as well as non-naturally occurring forms of RNA, including cRNA, antisense RNA, and any type of artificial (e.g., recombinant) transcript not endogenous to a cellular system. The term RNA also encompasses RNA molecules that comprise non-natural ribonucleotide analogues, such as 2-O-methylated ribonucleotides. RNA can be produced by any method, including by enzymatic synthesis or by artificial (chemical) synthesis. Enzymatic synthesis can include cell-free in vitro transcription systems and cellular systems, e.g., in a prokaryotic cell or in a eukaryotic cell.

The term “cDNA” refers to complementary or “copy” DNA. Generally cDNA is synthesized by a DNA polymerase having reverse transcriptase activity (e.g., a nucleic acid polymerase that uses an RNA template to generate a complementary DNA molecule) using any type of RNA molecule (e.g., typically mRNA) as a template. Alternatively, the cDNA can be obtained by directed chemical syntheses.

The terms “complementary” or “complementarity” refer to nucleic acid sequences capable of base-pairing according to the standard Watson-Crick complementary rules, or being capable of hybridizing to a particular nucleic acid segment under relatively stringent conditions. Optionally, nucleic acid polymers are complementary across only portions of their entire sequences. As used herein, the terms “complementary” or are used in reference to antiparallel strands of polynucleotides related by the Watson-Crick (and optionally Hoogsteen-type) base-pairing rules. For example, the sequence 5′-AGTTC-3′ is complementary to the sequence 5′-GMCT-3′. The terms “completely complementary” or “100% complementary” and the like refer to complementary sequences that have perfect Watson-Crick pairing of bases between the antiparallel strands (no mismatches in the polynucleotide duplex). The terms “partial complementarity,” “partially complementary,” “incomplete complementarity” or “incompletely complementary” and the like refer to any alignment of bases between antiparallel polynucleotide strands that is less than 100% perfect (e.g., there exists at least one mismatch in the polynucleotide duplex). Furthermore, two sequences are said to be complementary over a portion of their length if there exist one or more mismatch, gap or insertion in their alignment.

The term “gene” refers to a nucleic acid sequence encoding a gene product, The gene optionally comprises sequence information required for expression of the gene (e.g. promoters, enhancers, etc.). A “gene” is identified as an open reading frame (ORF) of preferably at least 50, 75, or 99 amino acids from which a messenger RNA is transcribed in the organism (e.g., if a single cell) or in some cell in a multicellular organism. The number of genes in a genome can be estimated from the number of mRNAs expressed by the organism, or by extrapolation from a well-characterized portion of the genome. When the genome of the organism of interest has been sequenced, the number of ORFs can be determined and mRNA coding regions identified by analysis of the DNA sequence. For example, the Saccharomyces cerevisiae genome has been completely sequenced and is reported to have approximately 6275 open reading frames (ORFs) longer than 99 amino acids. Analysis of these ORFs indicates that there are 5885 ORFs that are likely to specify protein products (Goffeau et al., 1996, Life with 6000 genes, Science 274: 546-567, which is incorporated by reference in its entirety for all purposes). In contrast, the human genome is estimated to contain approximately 35000 genes.

“Label” or “labeled” refers to incorporation of a detectable marker, for example by incorporation of a fluorophore, radiolabled compound or attachment to a polypeptide of moieties such as biotin that can be detected by the binding of a section moiety, such as marked avidin. Various methods of labeling polypeptide, nucleic acids, carbohydrates, and other biological or organic molecules are known in the art. Labels can be radioactive, fluorescent, chromagenic, chemiluminescent, or have other readouts or properties known in the art or later developed. Detection can be based on enzymatic activity, such as beta-galactosidase, beta-lactamase, horseradish peroxidase, alkaline phosphatase, luciferase; radioisotopes such as ³²P, ³H, ¹⁴C, ³⁵S, ¹²⁵I or ¹³¹I; fluorescent proteins, such as green fluorescent proteins; or other fluorescent labels, such as FITC, rhodamine, and lanthanides.

A “probe” is a nucleic acid probe having a single polynucleotide sequence. The term “synthetic probe” is used to indicate that the probe is produced by one or more synthetic or artificial manipulations, e.g., by chemical oligonucleotide synthesis, restriction digestion, amplification, cDNA synthesis, and the like.

The term “primer” refers to any nucleic acid that is capable of hybridizing at least at its 3′ end to a complementary or partially complementary nucleic acid molecule, where the free 3′ hydroxyl terminus is capable of being extended by a nucleic acid polymerase in a template-dependent manner.

According to the present invention, a “disease state” refers to any abnormal biological state of a cell. Thus the presence of a disease state may be identified by the same collection of biological constituents used to determine the cell's biological state. In general, a disease state will be detrimental to a biological system. A disease state may be a consequence of, inter alia, an environmental pathogen, for example a viral infection (e.g., AIDS, hepatitis B, hepatitis C, influenza, measles, etc.), a bacterial infection, a parasitic infection, a fungal infection, or infection by some other organism. A disease state may also be the consequence of some other environmental agent, such as a chemical toxin or a chemical carcinogen. As used herein, a disease state further includes genetic disorders wherein one or more copies of a gene is altered or disrupted, thereby affecting its biological function. Exemplary genetic diseases include, but are not limited to polycystic kidney disease, familial multiple endocrine neoplasia type I, neurofibromatoses, Tay-Sachs disease, Huntington's disease, sickle cell anemia, thalassemia, and Down's syndrome, as well as others (see, e.g., The Metabolic and Molecular Bases of Inherited Diseases, 7th ed., McGraw-Hill Inc., New York).

Other exemplary diseases include, but are not limited to, inflammation, septic shock, rheumatoid arthritis, acute pancreatitis, acute chest syndrome in patients with sickle cell disease, acute respiratory distress syndrome (ARDS), obesity, obesity-related insulin resistance, hyperalgesia, pulmonary edema, colitis, ischemia reperfusion, pleurisy, microbial infection, rheumatoid arthritis, skin inflammation, psoriasis, cancer, osteoporosis, asthma, autoimmune diseases, HIV, AIDS, rheumatoid arthritis, systemic lupus erythematosus, Type I insulin-dependent diabetes, tissue transplantation, malaria, African sleeping sickness, Chagas disease, toxoplasmosis, psoriasis, restenosis, inhibition of unwanted hair growth as cosmetic suppression, hyperparathyroidism, inflammation, treatment of peptic ulcer, glaucoma, Alzheimer's disease, suppression of atrial tachycardias, stimulation or inhibition of intestinal motility, Crohn's disease and other inflammatory bowel diseases, high blood pressure (vasodilation), stroke, epilepsy, anxiety, neurodegenerative diseases, hyperalgesic states, protection against hearing loss (especially cancer chemotherapy induced hearing loss), and pharmacological manipulation of cocaine reinforcement and craving in treating cocaine addiction and overdose and other fungal bacterial, viral, and parasitic diseases. In a specific embodiment, the disease, the level or progression of which is determined, or for which therapy is monitored according to the invention, is a genetic disease. Thus, in a specific embodiment, the disease is a cancer associated with a genetic mutation, e.g., translocation, deletion, or point mutation (for example, the Philadelphia chromosome).

In fact, with respect to the present invention, any biological state that is associated with a disease or disorder is considered to be a disease state. As used in the present invention, the “level” of a disease or disease state is an arbitrary measure reflecting the progression or state of a disease or disease state. Generally, a disease or disease state will progress through a plurality of levels or stages, wherein the affects of the disease become increasingly severe. Accordingly, a therapy or therapeutic regimen, as used herein, refers to a regimen of treatment intended to reduce or eliminate the symptoms of a disease. A therapeutic regimen will typically comprise, e.g., a prescribed dosage of one or more drugs.

Ideally, the effect of a therapy will be beneficial to a biological system in that it will tend to decrease the level of a disease state. However, in many instances, the effect of a therapy will be adverse to a biological system. For example, many therapies, such as drug regimens or chemotherapies, have toxic side effect. In such instances, it is important to monitor adverse effects so that the therapy may be adjusted, e.g., by reducing dosages or terminating the therapy altogether, before the adverse effects become too severe.

In general, a disease or disease state will have particular effects on the constituents of a biological system, i.e., “perturbations”. These effects can therefore be correlated to the level of the disease state. In particular, at least at low levels of disease state which comprise low levels of perturbation, individual diseases will generally mediate their effects through different, independent perturbations which can be independently correlated to a particular disease or disease state. Likewise, drugs or other agents which may be used in a therapy will each have unique perturbations on the state of a biological system which can be correlated to the level of efficacy of a particular therapy.

As used herein, the terms “array,” “microarray,” and “matrix” are interchangeable and mean a support, preferably solid, with nucleic acid probes attached to the support. Arrays and/or microarrays typically comprise a plurality of different nucleic acid probes that are coupled to a surface of a substrate in different, known locations. General arrays and microarrays are well-defined in the art.

As used herein, the term “Gel Electrophresis” means Separation of ionic molecules, (principally proteins or nucleic acids) by the differential migration through a gel according to the size, ionic charge, secondary structure or a combination thereof of the molecules in an electrical field. High resolution techniques normally use a gel support for the fluid phase. Examples of gels used are starch, acrylamide (for high resolution of short polynucleotide molecules), agarose or mixtures of acrylamide and agarose. Frictional resistance produced by the support causes size, rather than charge alone, to become the major determinant of separation. The gel may be in the form of a slab or capillary, which allows greater resolution. Smaller molecules with a more negative charge will travel faster and further through the gel toward the cathode of an electrophoretic cell when high voltage is applied. Similar molecules will group on the gel. They may be visualised by staining and quantitated, in relative terms, using densitometers which continuously monitor the photometric density of the resulting stain. The electrolyte may be continuous (a single buffer) or discontinuous, where a sample is stacked by means of a buffer discontinuity, before it enters the running gel/running buffer. The gel may be a single concentration or gradient in which pore size decreases with migration distance. The gel may also be denaturing to protein (e.g., contains SDS and/or reducing agents) or duplex nucleic acid (e.g., contains urea and/or formamide) or non-denaturing.

As used herein, the term “Native Gel” or “non-denaturing gel” means an electrophoresis gel that does not contain agents that cause the disruption of a molecule's native or active structure. For example, a non-denaturing gel would preserve the structure of a protein (e.g., secondary, tertiary or quaternary structure) or nucleic acid (e.g., duplex formation or single stranded nucleic acid secondary structure such as loops, hairpin structures, and the like).

In another aspect the invention relates to a computer database comprising 2-D matrix signatures generated using the methods of the invention from a plurality of organisms under a variety of physiological conditions. In a related aspect the invention includes a statistical based algorithm suitable for performing comparisons between a test or unknown signature and those contained in the database. For example, a computer algorithm could be used to analyze and compare regions of the reference signature and the test signature to determine the degree of statistical significance in identity of the reference to the test signatures. Examples of types of statistical analysis methods include ANOVA, MANOVA, ANCOVA, and MANCOVA that can be used to determine the significance of differences between samples.

The present invention also provides methods for detecting/identifying the presence of a particular nucleic acid source. For example, the method of this aspect of the invention comprises using the 2-D oligonucleotide matrix and the methods of the invention to generate a database of “reference signatures” based on input nucleic acid from many different biological sources, for example, virus, bacteria, fungi, yeast, unicellular organisms, plants, and animals. Next, nucleic acid is isolated from an unknown or unidentified source and suitably labeled. Using the methods of the invention, a “test signature” is generated using the nucleic acid. Lastly, the test signature is compared to the database of reference signatures to determine the identity of the organism. This method is particularly useful, for example, in the detection/identification of a potential pathogenic organism such as a virus or a bacteria.

Measurement of the transcriptional state of a cell is preferred in this invention, not only because it is relatively easy to measure but also because, even though a protein of interest may not directly modulate transcription, even the slight disruption of protein activity in a cell almost always results in a measurable change, through direct or indirect effects, in the transcriptional state. A reason that disruption in a protein's activity level changes the transcriptional state of a cell is because the previously mentioned feedback systems, or networks, which react in a compensatory manner to infections, genetic modifications, environmental changes, drug administration, and so forth do so primarily by altering patterns of gene expression or transcription. As a result of internal compensations, many perturbations to a biological system, although having only a muted effect on the external behavior of the system, can nevertheless profoundly influence the internal response of individual elements, e.g., gene expression, in the cell.

Microarrays or arrays are known in the art and consist of a surface to which probes that correspond in sequence to gene products (e.g., cDNAs, mRNAs, cRNAs, polypeptides, and fragments thereof), can be specifically hybridized or bound at a known position. A discussion of microarrays, methods and techniques, can be found in the following references, Schena et al., 1995, Quantitative monitoring of gene expression patterns with a complementary DNA microarray, Science 270: 467-470; DeRisi et al., 1996, Use of a cDNA microarray to analyze gene expression patterns in human cancer, Nature Genetics 14: 457-460; Shalon et al., 1996, A DNA microarray system for analyzing complex DNA samples using two-color fluorescent probe hybridization, Genome Res. 6: 639-645; and Schena et al., 1995, Parallel human genome analysis; microarray-based expression of 1000 genes, Proc. Natl. Acad. Sci. USA 93: 10539-11286; Sambrook et al., Molecular Cloning—A Laboratory Manual (2nd Ed.), Vol. 1-3, Cold Spring Harbor Laboratory, Cold Spring Harbor, N.Y., 1989. Each of the aforementioned articles is incorporated by reference in its entirety for all purposes.

In one embodiment, the each position in the universal oligonucleotide matrix represents a discrete binding site for a product encoded by a gene (e.g., a protein or RNA), and in which binding sites are present for products of most or almost all of the genes in the organism's genome. In a preferred embodiment, the “binding site” (hereinafter, “site”) is comprised of the oligonucleotide probe or oligonucleotide analog of the invention to which a particular cognate cDNA can specifically hybridize.

The methods of this invention determine the level (e.g., the stage or progression) of one or more disease states of a subject and, more specifically, detect changes in the biological state of a subject which are correlated to one or more disease states. The methods of the present invention are also applicable to monitoring the disease state or states of a subject undergoing one or more therapies. Thus, the present invention also provides methods for determining or monitoring efficacy of a therapy or therapies (i.e., determining a level of therapeutic effect) upon a subject. In a specific embodiment, the methods of the invention can be used to assess therapeutic efficacy in a clinical trial, e.g., as an early surrogate marker for success or failure in such a clinical trial.

Aspects of the biological state of a cell of a subject, for example, the transcriptional state, the translational state, or the activity state, are measured as described above. The collection of these measurements, optionally graphically represented, is called herein the “diagnostic signature”. Aspects of the biological state of a cell which are similar to those measured in the diagnostic profile, e.g., the transcriptional state, are measured in an analogous subject or subjects in response to a known correlated disease state or, if therapeutic efficacy is being monitored, in response to a known, correlated effect of a therapy. The collection of these measurements, optionally graphically represented, is called herein the “test or response signature.” In cases where therapeutic efficacy is to be monitored, the response signature may be correlated to a beneficial effect, an adverse effect, such as a toxic effect, or to both beneficial and adverse effects.

More generally, the methods of the present invention allow one to monitor a plurality of disease states or therapies in an individual subject; for example in a subject having several genetic mutations that are each associated with a particular disease, or in a subject undergoing several therapeutic regimes simultaneously (for example, a patient taking several drugs, each of which has a different effect). Accordingly, diagnostic and response signatures are obtained individually for each disease or therapy.

In a preferred embodiment, comparison of a diagnostic signature with response signatures is performed by a method in which an objective measure of difference between a diagnostic profile and a response profile determined for some perturbation level, i.e., for some level of disease or therapeutic efficacy, is minimized. The measurements can be performed by standard techniques of numerical analysis. See, e.g., Press et al., 1996, Numerical Recipes in C, 2nd Ed. Cambridge Univ. Press, Ch. 10; Branch et al., 1996, Matlab Optimization Toolbox User's Guide, Mathworks (Natick, Mass.).

In a preferred embodiment the present invention makes use of a universal oligonucleotide matrix or array for analyzing the transcriptional state in a cell, and especially for measuring the transcriptional states of cells exposed to graded levels of a therapy of interest such as graded levels of a drug of interest or to graded levels of a disease state of interest. Arrays are produced by hybridizing detectably labeled polynucleotides representing the mRNA transcripts present in a cell (e.g., fluorescently labeled cDNA synthesized from total cell mRNA) to a probe array. A given binding site or unique set of binding sites in the array will specifically bind the product of a single gene in the cell. There may be more than one physical binding site (hereinafter “site”) per specific mRNA.

It will be appreciated that when cDNA complementary to the RNA of a cell is made and hybridized to an array under suitable hybridization conditions, the level of hybridization to the site in the array corresponding to any particular gene will reflect the prevalence in the cell of mRNA transcribed from that gene. For example, when detectably labeled (e.g., with a fluorophore) cDNA complementary to the total cellular mRNA is hybridized to an array, the site on the array corresponding to a gene (i.e., capable of specifically binding the product of the gene) that is not transcribed in the cell will have little or no signal (e.g., fluorescent signal), and a gene for which the encoded mRNA is prevalent will have a relatively strong signal.

In the case of therapeutic efficacy (e.g., in response to drugs) one cell is exposed to a therapy and another cell of the same type is not exposed to the therapy. In the case of disease states one cell exhibits a particular level of disease state and another cell of the same type does not exhibit the disease state (or the level thereof). The cDNA derived from each of the two cell types are labeled and each is hybridized to the matrix of the invention and their signatures are then compared. In one embodiment, for example, cDNA from a cell treated with a drug (or exposed to a pathway perturbation) is synthesized using labeled dNTP, and cDNA from a second cell, not drug-exposed, is synthesized using a labeled dNTP. When the two cDNAs are hybridized to the 2-D matrix, the relative signature is determined for each, and any relative difference is detected. However, it will be recognized that it is also possible to use cDNA from a single cell, and compare, for example, the mRNA in, e.g., a therapy-exposed or diseased cell and an untreated or nondiseased cell.

In a preferred embodiment the universal matrix of the invention contains binding sites for products of at least 70% of all genes in the target organism's genome, most preferably the array contains binding sites for 99.9% of all genes in the target organism's genome. Preferably, the array has binding sites for genes relevant to the action of a drug of interest or in a biological pathway of interest.

### Preparing Nucleic Acids for Use in the Matrix

As noted above, the “binding site” to which a particular cognate cDNA specifically hybridizes is usually a nucleic acid or nucleic acid analogue attached at that binding site. In one embodiment, the binding sites of the oligonucleotide matrix are DNA polynucleotides corresponding to at least a portion of each gene in an organism's genome.

Computer programs are useful in the design of PCR primers with the required specificity and optimal amplification properties. See, e.g., Oligo version 5.0 (National Biosciences). PCR methods are well known and are described, for example, in Innis et al. eds., 1990, PCR Protocols: A Guide to Methods and Applications, Academic Press Inc. San Diego, Calif., which is incorporated by reference in its entirety for all purposes. It will be apparent that computer controlled robotic systems are useful for isolating and amplifying nucleic acids.

Generating the polynucleotides for the microarray is by synthesis of synthetic polynucleotides or oligonucleotides, e.g., using N-phosphonate or phosphoramidite chemistries (Froehler et al., 1986, Nucleic Acid Res 14: 5399-5407; McBride et al., 1983, Tetrahedron Lett. 24: 245-248). Synthetic sequences are between about 15 and about 500 bases in length, more typically between about 20 and about 50 bases. In some embodiments, synthetic nucleic acids include non-natural bases, e.g., inosine. As noted above, nucleic acid analogues may be used as binding sites for hybridization. An example of a suitable nucleic acid analogue is peptide nucleic acid (see, e.g., Egholm et al., 1993, PNA hybridizes to complementary oligonucleotides obeying the Watson-Crick hydrogen-bonding rules, Nature 365: 566-568; see also U.S. Pat. No. 5,539,083).

### Attaching Nucleic Acids to a Solid Surface

The nucleic acid or analogue are attached to a solid support, which may be made from glass, plastic (e.g., polypropylene, nylon), polyacrylamide, nitrocellulose, or other materials. Techniques are known for producing arrays containing thousands of oligonucleotides complementary to defined sequences, at defined locations on a surface using photolithographic techniques for synthesis in situ (see, Fodor et al., 1991, Light-directed spatially addressable parallel chemical synthesis, Science 251: 767-773; Pease et al., 1994, Light-directed oligonucleotide arrays for rapid DNA sequence analysis, Proc. Natl. Acad. Sci. USA 91: 5022-5026; Lockhart et al., 1996, Expression monitoring by hybridization to high-density oligonucleotide arrays, Nature Biotech 14: 1675; U.S. Pat. Nos. 5,578,832; 5,556,752; and 5,510,270; Blanchard et al., 1996, High-Density Oligonucleotide arrays, Biosensors & Bioelectronics 11: 687-90; Maskos and Southern, 1992, Nuc. Acids Res. 20: 1679-1684); Sambrook et al., Molecular Cloning—A Laboratory Manual (2nd Ed.), Vol. 1-3, Cold Spring Harbor Laboratory, Cold Spring Harbor, N.Y., 1989, each of which is incorporated by reference in its entirety for all purposes).

### Generating Labeled Probes

Methods for preparing total and poly(A)⁺ RNA are well known and are described generally in Sambrook et al., supra. In one embodiment, RNA is extracted from cells of the various types of interest in this invention using guanidinium thiocyanate lysis followed by CsCl centrifugation (Chirgwin et al., 1979, Biochemistry 18: 5294-5299). Poly(A)⁺ RNA is selected by selection with oligo-dT cellulose (see Sambrook et al., supra). Cells of interest include wild-type cells, drug-exposed wild-type cells, modified cells, and drug-exposed modified cells.

Labeled cDNA is prepared from mRNA by oligo dT-primed or random-primed reverse transcription, both of which are well known in the art (see e.g., Klug and Berger, 1987, Methods Enzymol. 152: 316-325). Reverse transcription may be carried out in the presence of a dNTP conjugated to a detectable label, most preferably a fluorescently labeled dNTP. Alternatively, isolated mRNA can be converted to labeled antisense RNA synthesized by in vitro transcription of double-stranded cDNA in the presence of labeled dNTPs (Lockhart et al., 1996, Expression monitoring by hybridization to high-density oligonucleotide arrays, Nature Biotech. 14: 1675, which is incorporated by reference in its entirety for all purposes). In alternative embodiments, the cDNA or RNA probe can be synthesized in the absence of detectable label and may be labeled subsequently, e.g., by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent.

When fluorescently-labeled probes are used, many suitable fluorophores are known, including fluorescein, lissamine, phycoerythrin, rhodamine (Perkin Elmer Cetus), Cy2, Cy3, Cy3.5, Cy5, Cy5.5, Cy7, FluorX (Amersham) and others (see, e.g., Kricka, 1992, Nonisotopic DNA Probe Techniques, Academic Press San Diego, Calif.). It will be appreciated that pairs of fluorophores are chosen that have distinct emission spectra so that they can be easily distinguished.

In another embodiment, a label other than a fluorescent label is used. For example, a radioactive label, or a pair of radioactive labels with distinct emission spectra, can be used (see Zhao et al., 1995, High density cDNA filter analysis: a novel approach for large-scale, quantitative analysis of gene expression, Gene 156: 207; Pietu et al., 1996, Novel gene transcripts preferentially expressed in human muscles revealed by quantitative hybridization of a high density cDNA array, Genome Res. 6: 492). However, because of scattering of radioactive particles, and the consequent requirement for widely spaced binding sites, use of radioisotopes is a less-preferred embodiment.

In one embodiment, labeled cDNA is synthesized by incubating a mixture containing 0.5 mM dGTP, dATP and dCTP plus 0.1 mM dTTP plus fluorescent deoxyribonucleotides (e.g., 0.1 mM Rhodamine 110 UTP (Perken Elmer Cetus) or 0.1 mM Cy3 dUTP (Amersham)) with reverse transcriptase (e.g., SuperScript™ II, LTI Inc.) at 42° C. for 60 min.

### Hybridization

Nucleic acid hybridization and wash conditions are chosen so that the probe “specifically binds” or “specifically hybridizes” to a specific array site, i.e., the probe hybridizes, duplexes or binds to a sequence array site with a complementary nucleic acid sequence but does not hybridize to a site with a non-complementary nucleic acid sequence. As used herein, one polynucleotide sequence is considered complementary to another when, if the shorter of the polynucleotides is less than or equal to 25 bases, there are no mismatches using standard base-pairing rules or, if the shorter of the polynucleotides is longer than 25 bases, there is no more than a 5% mismatch. Preferably, the polynucleotides are perfectly complementary (no mismatches). It can easily be demonstrated that specific hybridization conditions result in specific hybridization by carrying out a hybridization assay including negative controls (see, e.g., Shalon et al., supra, and Chee et al., supra).

Optimal hybridization conditions will depend on the length (e.g., oligomer versus polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., 1987, Current Protocols in Molecular Biology, Greene Publishing and Wiley-Interscience, New York, which is incorporated in its entirety for all purposes. When the cDNA microarrays of Schena et al. are used, typical hybridization conditions are hybridization in 5×SSC plus 0.2% SDS at 65° C. for 4 hours followed by washes at 25° C. in low stringency wash buffer (1×SSC plus 0.2% SDS) followed by 10 minutes at 25° C. in high stringency wash buffer (0.1×SSC plus 0.2% SDS) (Shena et al., 1996, Proc. Natl. Acad. Sci. USA, 93: 10614). Useful hybridization conditions are also provided in, e.g., Tijessen, 1993, Hybridization With Nucleic Acid Probes, Elsevier Science Publishers B.V. and Kricka, 1992, Nonisotopic DNA Probe Techniques, Academic Press San Diego, Calif.

### Signal Detection and Data Analysis

When fluorescently labeled probes are used, the fluorescence emissions at each site of a transcript array can be, preferably, detected by scanning confocal laser microscopy. Alternatively, a laser can be used that allows simultaneous specimen illumination at wavelengths specific to the fluorophore and simultaneous analysis (see Shalon et al., 1996, A DNA microarray system for analyzing complex DNA samples using two-color fluorescent probe hybridization, Genome Research 6: 639-645, which is incorporated by reference in its entirety for all purposes). In a preferred embodiment, the arrays are scanned with a laser fluorescent scanner with a computer controlled X-Y stage and a microscope objective. Fluorescence laser scanning devices are described in Schena et al., 1996, Genome Res. 6: 639-645 and in other references cited herein. Alternatively, the fiber-optic bundle described by Ferguson et al., 1996, Nature Biotech. 14: 1681-1684, may be used to monitor mRNA abundance levels at a large number of sites simultaneously. Signals are detected, for example, by a CCD camera, and recorded. In a preferred embodiment the invention comprises a computer for analysis of the signatures, e.g., using a 12 bit analog to digital board. In one embodiment the scanned image is despeckled using a graphics program (e.g., Hijaak Graphics Suite) and then analyzed using an image gridding program that creates a spreadsheet of the average hybridization at each wavelength at each site.

In one embodiment of the invention, signatures or profiles are generated that reflect the transcriptional state of a cell of interest. The signatures are made by hybridizing a mixture of labeled probes corresponding (i.e., complementary) to the mRNA of a cell of interest, to the universal oligonucleotide matrix according to the methods of the present invention.

Diagnostic signatures may be obtained for any cell type in which it may be desirable to analyze the level of some disease state or of some therapeutic effect. Preferably, the disease state or therapy must be one for which response signatures can be generated. Cells for which it may be desirable to obtain diagnostic signatures include, for example, cells of a patient suspected of having a level of a disease state associated with one or more genetic mutations, as well as cells of a patient which has been exposed to a drug or a combination of drugs or other therapies and which exhibit a level of therapeutic effect.

To measure diagnostic signatures, e.g., of cells exposed to a drug (or to some other therapy), the cells, or the organism/patient from which the cells are obtained, are exposed to some level of the drug of interest, preferably a level corresponding to clinical dosages of the drug, and a measure of therapeutic effect is taken (e.g., measuring change in amount of a disease marker or disease symptom). When the cells are grown in vitro, the drug is usually added to their nutrient medium. In the case of yeast, it is preferable to harvest the yeast in early log phase, since expression patterns are relatively insensitive to time of harvest at that time. The drug is added is a graded amount that depends on the particular characteristics of the drug. In some cases a drug will be solubilized in a solvent. The cells exposed to therapy or having a disease state and cells not exposed to therapy and/or not having a disease state (or the particular level of disease state) are used to construct diagnostic signatures.

In certain aspects, the invention relates to methods for performing analysis of complex samples of nucleic acids such as genomic DNA. The invention is not limited to any particular type of nucleic acid sample and, as such, is applicable to any of plant, bacteria, and animal (including human). The genomic DNA may be isolated according to methods known in the art, such as PCR, reverse transcription, and the like. It may be obtained from any biological or environmental source, including plant, animal (including human), bacteria, fungi or algae. Any suitable biological sample can be used for assay of genomic DNA. Convenient suitable samples include whole blood, tissue, semen, saliva, tears, urine, fecal material, sweat, mucus, skin and hair.

Additional advantages and benefits will be apparent to those of skill in the art from the following examples with reference to the drawings.

### EXAMPLES

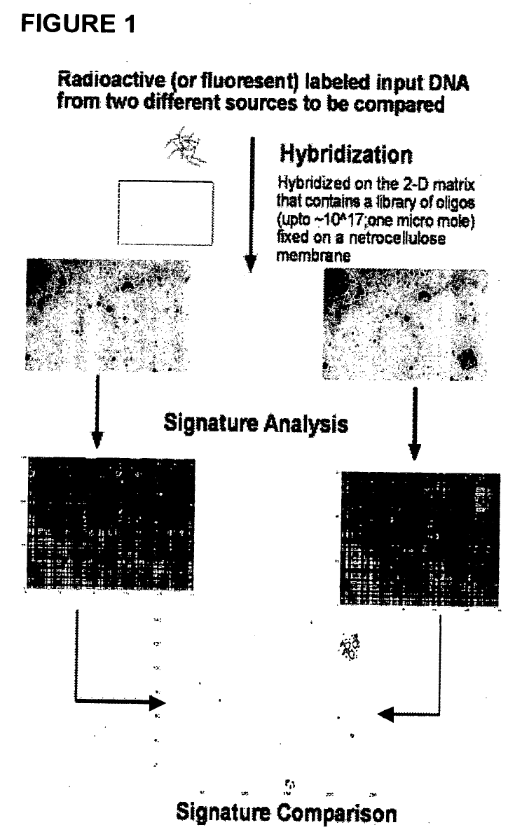

The universal oligonucleotide matrix is a membrane with large number of oligonucleotides immobilized on the surface. It is constructed by a method consisting of separating a large library of DNA oligos with up to more than 1017 different oligonucleotides having from 30 to 60 nucleotides in length. The universal oligonucleotide matrix is capable of monitoring or profiling the existence of any input biological or non-biological DNA or RNA samples with high sensitivity and capacity due to the large number of probes on the surface. For example, human genomic DNA or total RNAs can be used as the input, and the hybridization signature of these nucleic acids to the matrix is the output that represents the genomic “signature” of this individual (FIG. 1). Due to the high sensitivity and capacity of the universal oligonucleotide matrix, potentially it could detect minor differences of any closely related input DNAs or RNAs. Therefore, it generates genomic, gene expression or total RNA profile signatures of specific organism, cell type, or tissue that are indicative of different species, phenotypes, or diseases. Similarly, human genomic DNA of different race, gender, age, and other traits should generate distinctive signatures.

In addition, analysis of information obtained is based upon the recognition of patterns of hybridizations to the huge DNA library immobilized in the matrix. Thus, the information processing task is to distinguish between patterns from different samples. This alleviates errors from background noise and mis-hybridization. In certain aspects the tools used for analysis are trainable in order to customize the user's application. Because, the matrix is universal, applications such as environmental monitoring, medical diagnostics, and bio-defense monitoring are just few of many potential applications.

### Example 1

### Universal Oligonucleotide Matrix

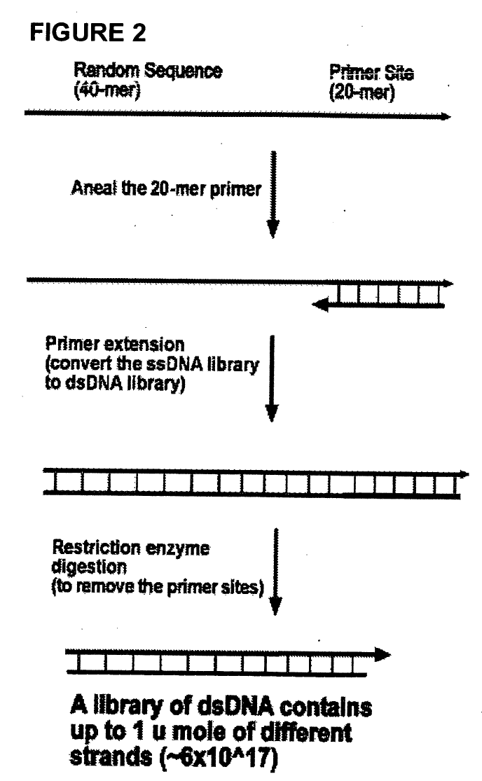

The first step in building the universal oligonucleotide matrix with up to 10¹⁷ oligos having from about 30 to 60 nucleotides is to prepare the duplex DNA library contains that many different sequences. As illustrated in FIG. 2, ssDNA oligonucleotides are synthesized having (in this example) 60 nucleotides-40 nucleotides of designed random sequences at its 5′ end and a 20-mer long fixed sequence at the 3′ end that is served as the primer site to convert this machine synthesized ssDNA-library to a dsDNA-library. The number of the different sequences in each library is limited by the DNA synthesizer machine. The current limitation for each synthesis of most DNA synthesizer is about one micro mole which is about 6×10¹⁷ DNA molecules.

There is a great degree of freedom in the design of the contents of each oligonucleotide library. As such, in certain embodiments, the matrix includes the use of oligo libraries that contain variations in sequence due to alterations in the synthesis protocol used. For example, if the DNA synthesizer machine was programmed to incorporate all the four possible nucleotides (¼ each of A, T, C, G) in every other position within the 5′ end 40-mer region. In a micro mole scale synthesis, it would generate a library of ssDNA that have 4²⁰ different sequences (˜10¹²) with more than 100,000 (10⁵) copies each (assumed the total amount of DNA synthesized theoretically is 1 μmole; ˜6×10¹⁷).

As shown in FIG. 2, the synthesized ssDNA library can then be converted to dsDNA by a simple primer extension reaction using DNA polymerase. The 20-bp primer region in each duplex DNA can then be cut off by a restriction enzyme digestion that recognize a pre-designed restriction site close to the end of the primer site. The result of these steps would be a library of dsDNA with the desired different sequences.

Construction of the universal oligonucleotide matrix using the dsDNA oligo library

1. Separation on a Denaturing Gradient Gel Electrophoresis System

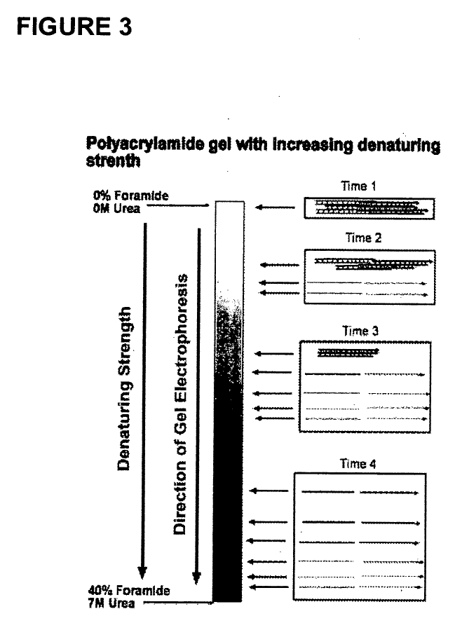

To construct the universal oligonucleotide matrix, the dsDNA-library is subjected to electrophoresis through a denaturing gradient gel. On this gel electrophoresis system, the denaturing strength inside the polyacrydamide gel (10%) changes continuously from 0% strength to 100% strength (defined as about 7 M urea plus about 40% formamide) from top to bottom. The principle of this system is shown in FIG. 3, here the 40-bp dsDNA library (as described in FIG. 2) are loaded on top of he gel, once the power is on, the electrophoresis (run in a buffer containing, for example, Tris, EDTA, Acetic Acids, and Mg²⁺) will start to move the duplex DNAs into the gel. The whole population of the dsDNAs begin to encounter increasing chemical denaturing strength (from urea and formamide) that breaks the hydrogen bonds between the two single strands of each duplex DNA molecules that have same length but different sequences, thus, each duplex DNA molecule has a unique melting temperature. At the beginning of the electrophoresis (for example, Time 1 in FIG. 3), the whole population of duplex DNAs would travel as double-stranded molecules, therefore at about the same speed. However, once they travel further, they will encounter greater denaturing strength from the gel. Those duplex DNAs that have lowest melting temperature will start to denature, result in the separation of the duplex to two single strands. Those molecules that denatured travel as single-stranded DNAs afterward, thus migrating in the gel much faster than the rest of un-denatured duplex DNA population (Time 2, and Time 3 in FIG. 3). With 6 imaginative duplex DNAs of same length but having from low to high melting temperatures (light to darker blues) as example, the right panel of FIG. 3 illustrates this principle of the separation of the DNA library.

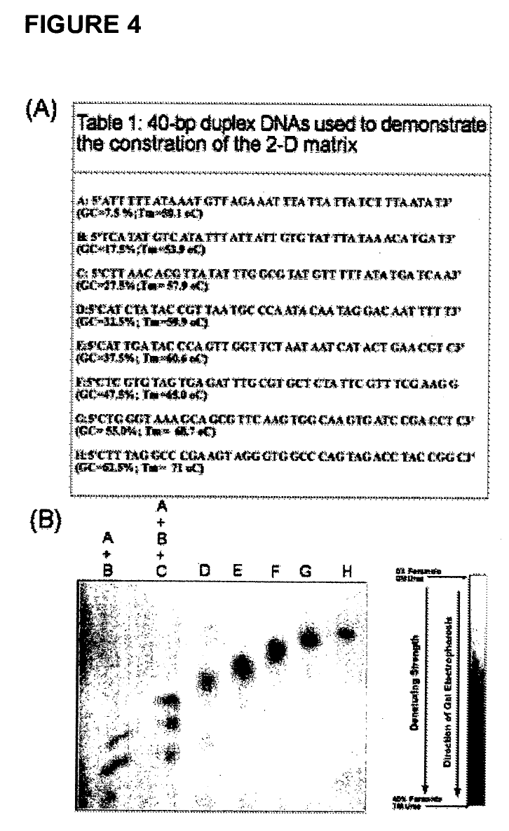

Using specially designed 40-bp duplex DNA sequences (some of the representative sequences shown in FIG. 4 (A)), the denaturing gradient gel electrophoresis can separate duplex DNAs of same length according to their melting temperatures, and this method is very precise and reproducible.

In the example in FIG. 4, the denaturing gradient gel electrophoresis system separates eight designed 40-bp duplex DNA markers (sequences in FIG. 4A) that have increasing GC contents (melting temperatures) in a denaturing gradient gel (10% polyacrylamide in TAE plus 10 nM mg++; FIG. 4B).

The results indicate the 8 duplex DNAs with the same length can be separated according to their GC contents (melting temperatures; FIG. 4B). Repeated experiments indicate that the duplex A which has the lower melting temperature always migrate fastest in the denaturing gradient followed by B, C, then, D and so on. The H strand always migrate the slowest when compared to the rest because it has highest melting temperature. The orders of the migrations of these duplexes are also very reproducible. For example, the left most lane of the gel contains mixture of samples A plus B, and the next lane contains samples A, plus B and C. So in these two lanes, samples A and B are clearly migrating to the same positions.

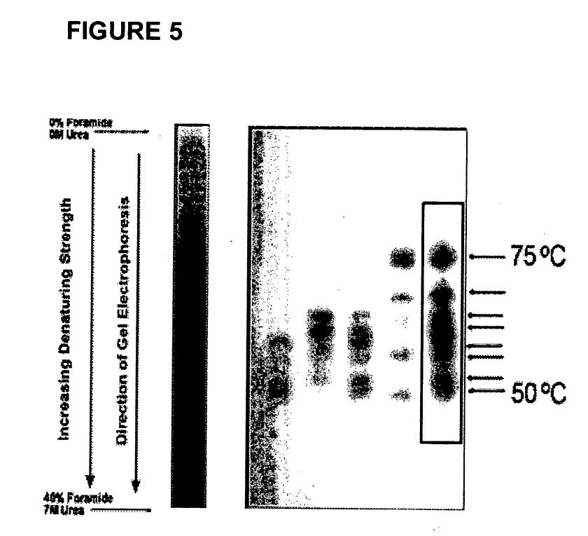

To further characterize the denaturing gradient gel electrophoresis system, another 7 different 40-bp duplex DNAs were designed that have similar distribution of the melting temperature ranges. FIG. 5 shows the separation according to melting temperatures of different combinations of these 15 duplex DNAs. The denaturing gradient gel system is less efficient at separating duplex DNA molecules that have similar melting temperatures but differing sequences, because the principle of the separation is based on the fact that each duplex DNA of same length should have a unique melting temperature and would be melted at its unique melting temperature. Therefore, in the last lane of FIG. 5, when all the 15 samples are mixed in one lane, one cannot detect 15 distinguish bands on the gel (i.e., several of them have almost identical melting temperatures as shown in Table 2). Instead we can observe about 8 to 9 distinct bands represent the different groups of melting temperatures in the 15 samples.

TABLE 2
Sample DNAs with Increasing Melting Temperatures.

| | | Melting |
|---|---|---|
| Oligonucleotide Sequence | C % | Temperature |
| 1. 5′-ATTTTTATAAATGTTAGAAATTTATTATTATCTTTAATAT | .5% | 50.1° C. |
| (SEQ ID NO:9) | | |
| 2. 5′-TTATTCAAAATATTTTCTATTATATTTAAACATATAAATT | .5% | 50.2° C. |
| (SEQ ID NO:10) | | |
| 3. 5′-TCATATGTCATATTTATTATTGTGTATTTATAAACATGAT | 7.5% | 53.9° C. |
| (SEQ ID NO:11) | | |
| 4. 5′-GATAATTTTGCCAGAGAAATAATATTCAAATAAAATGAAT | 0.0% | 55.3° C. |
| (SEQ ID NO:12) | | |
| 5. 5′-CTTAACACGTTATATTTGGCGTATGTTTTTATATGATCAA | 7.5% | 57.9° C. |
| (SEQ ID NO:13) | | |
| 6. 5′-CATCTATACCGTTAATGCCCAATACAATAGGACAATTTTT | 2.5% | 59.5° C. |
| (SEQ ID NO:14) | | |
| 7. 5′-TTTAACATGATTCTTGTGTGGGTTGTGTATATCGCATCTA | 5.0% | 64.1° C. |
| (SEQ ID NO:15) | | |
| 8. 5′-CATTGATACCCAGTTGGTTCTAATAATCATACTGAACGTC | 7.5% | 60.6° C. |
| (SEQ ID NO:16) | | |
| 9. 5′-AGTAGGTGAGATAGAGGAGAACTCAGACTCATGGTAAGCT | 5.0% | 64.1° C. |
| (SEQ ID NQ:17) | | |
| 10. 5′-TGCATTCAATCTCGTGTCAATGGAGAAGACACGACTGGTG | 7.5% | 66.2° C. |
| (SEQ ID NO:18) | | |
| 11. 5′-CTCGTGTAGTGAGATTTGCGTGCTCTATTCGTTTCGAAGG | 7.5% | 65.0° C. |
| (SEQ ID NO:19) | | |
| 12. 5′-CTCAGGATCTTCATGCTACGCCCAGCCGATGTACGGTTGA | 5.0% | 68.9° C. |
| (SEQ ID NO:20) | | |
| 13. 5′-CTGGGTAAAGCAGCGTTCAAGTGGCAAGTGATCCGACCTC | 5.0% | 68.7° C. |
| (SEQ ID NO:21) | | |
| 14. 5′-CTTTAGGCCCGAAGTAGGGTGGCCCAGTAGACCTACCGGC | 2.5% | 71.0° C. |
| (SEQ ID NO:22) | | |
| 15. 5′-GGCCCGGCTGCGGGTACTTGAACCTGACTCTTGCCGGGCC | 0.0% | 75.4° C. |
| (SEQ ID NO:23) | | |

To test the gradient denaturing gel system in separating large number of different DNAs with different sequences, several duplex DNA libraries were constructed with increasing complexity. These libraries were designed from a single 40-nucleotide template sequence. Based on this template sequence, 5 different pools of DNAs were made with 4, 8, 12, 16, and 20 out of the 40 positions containing 2 possible nucleotides (Table 3).

TABLE 3

| | |
|---|---|
| Names | Sequences |

| | |
|---|---|
| Template | 5′-ACT ATA ACA AAA CCG CTG AGC ACA CAC TGT GAC GTG GCC G |
| (SEQ ID NO:24) | |
| 4M | 5′-ACT ATA ACA RAA CCG YTG AGC ACR CAC TGT RAC GTG GCC G |
| 8M | 5′-ACY ATA AYA AAA YCG CTR AGC AYA CAC YGT GAY GTG GYC G |
| 12M | 5′-ACT RTA RCA RAA YCG YTG RGC RCA YAC YGT RAC RTG RCC G |
| 16M | 5′-AYT RTA RCA RAR CYG YTR ARC RCA YAC YGT RAC RTG RCY G |
| 20M | 5′-AYT RTR AYA RAR CYG YTR ARC RCR CRC YGY GRC RTR GYC R |

Note:
R = A or G, Y = C or T

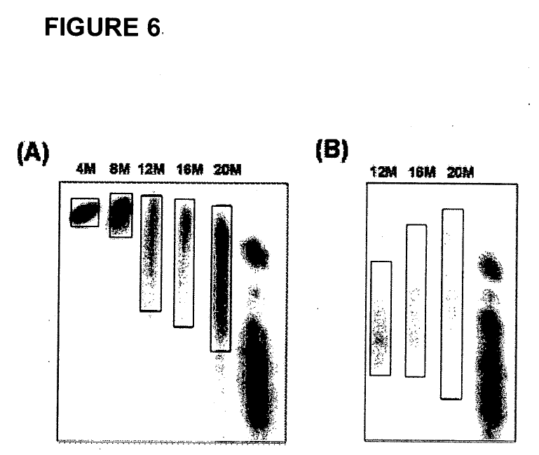

Thus, the library labeled 4M contains 2⁴ (16) different DNA sequences, and the one labeled 20M contains 2²⁰ (1,048,576) different sequences. These samples were run side by side in the gradient denaturing gel system. As expected, the results indicate as the number of different sequences in the library increased, a much wider range of melting temperatures of the DNA molecules is observed. The DNAs in the library separated in a larger area and quite evenly in the gel, indicated by a long smear marked by the rectangles (FIG. 6A). Similar result was obtained when another set of DNA libraries was tested (FIG. 6B).

Construction of the Universal Oligonucleotide Matrix, “Bio-Profiler” Membrane

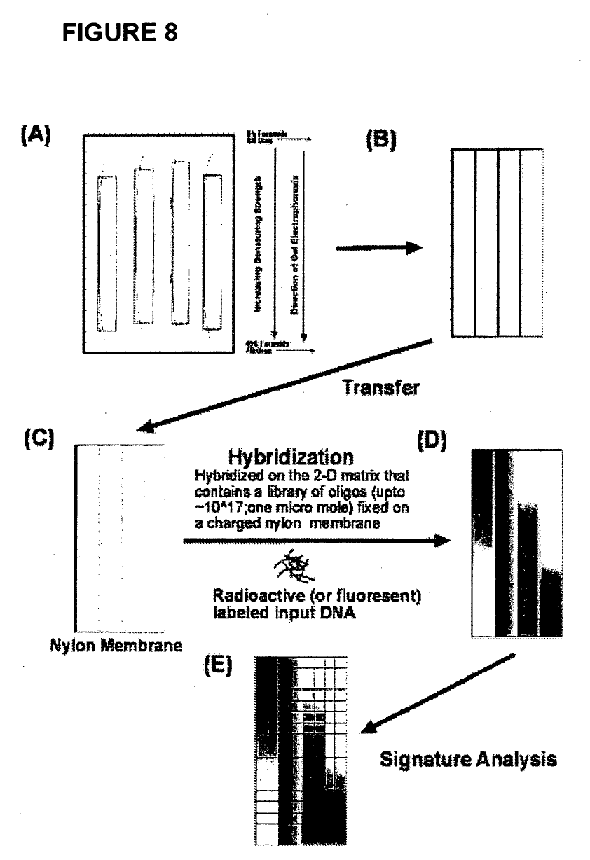

FIG. 8 diagrams the principle of making a universal oligonucleotide matrix membrane. The main idea is based on the fact that a simple chemical denaturing gradient gel electrophoresis system, as those shown in FIGS. 3-6, is capable of separating a large population of easy to make oligonucleotides converted duplex DNAs of same length according to their melting temperatures. Most importantly, experiments have shown that the DNAs in this system behave consistently and reproducibly, the DNAs with lower melting temperatures always denatured before those have higher melting temperatures. In one embodiment of the universal oligonucleotide matrix of the invention, many DNA libraries are separated, each can contains up to 1017 different sequences, in the denaturing gradient gel system as shown in FIG. 8A. After staining the gel (with ethidium bromide or other reagents), we can then cut the area containing the DNAs from the gel (indicated by the red box in FIG. 8A).

Next, the cut out gel strips are aligned and transferred to a commercially available membrane such as super charged nylon from Schleicher& Schuell by standard blotting methods. The resulting nylon membrane now has the DNAs from the libraries attached on the surface can be used to probe any radioactive or fluorescent labeled input DNA or RNA samples (FIG. 8D). The signal generated presents the signature of the input nucleic acids (FIG. 8E).

As shown in FIG. 8A, each lane in the gradient denaturing gel can be loaded with an independent DNA library that in theory can have up to 1 μmole (˜6×10¹⁷) of different sequences. However, if each lane contains too many different sequences, the chance for the DNA molecules that have different sequences but same melting temperature would be higher. These molecules would run at the same position in the denaturing gradient gel system that might hinder the detection of the hybridization signal. On the other hand, there is no limitation of the number of lanes (libraries) from the gel to be used to make the matrix; it could be as many as thousands or more.

An example, using the method described in FIG. 8, we have produced a matrix with 8 DNA libraries each containing about 1 million different 40-mer oligos (220; sequences shown in Table 4).

TABLE 4
Sequence of 8 libraries.

| Names | Sequences |
|---|---|
| L1 | 5′- |
| | AKTKTTMTMMAKGKTAKAMMTKTMTTMTKMTKTKKAMTMT-3′ |
| L2 | 5′- |
| | TMAKATKTMMTMTKTAKTMKTKTKTAKTKMTMAMMAKGMT-3′ |
| L3 | 5′- |
| | CKTMACMCKKTMTMTTKGKMGKAKGTKTKKAKAKKAKCMA-3′ |
| L4 | 5′- |
| | CMTMTAKAMMGKTMATKCMMAMTMCAMTMKGMCMMTKTKT-3′ |
| L5 | 5′- |
| | CMTKGAKAMMCMGKTGKTKMTMAKAAKCMKAMTKMAMGKC-3′ |
| L6 | 5′- |
| | CKCKTGKAKKGMGMTTKGMKTKCKCTMTKMGKTKMGMAKG-3′ |
| L7 | 5′- |
| | CKGKGTMAMKCMGMGTKCMMGKGKCAMGKKAKCMKAMCKC-3′ |
| L8 | 5′- |
| | CKTKAGKCMMGMAMTAKGKKGKCMCAKTMKAMCKMCMGKC-3′ |

Note:
M = (A or C), K = (G or T)

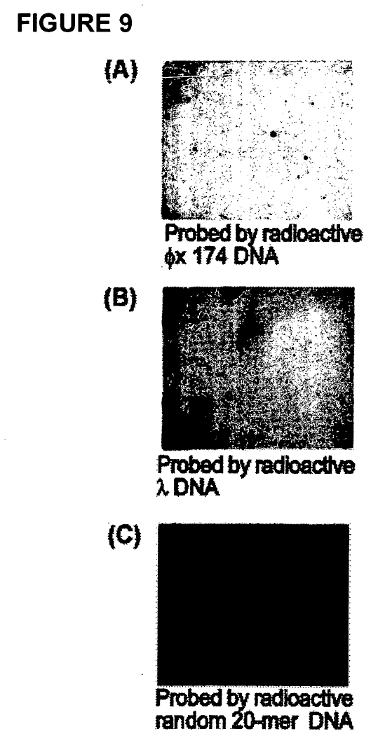

Thus, the total 40-mers in this matrix would have about 8 million different 40-mers as probes. The nylon membrane was probed with 3 different probes that have increasing complexities. In the first experiment, the matrix was probed by radioactive probes using φX 174 DNA (˜4 kb) as template. As shown in FIG. 9A, several spots in the matrix indicate hybridization. After being stripped of the probes, the same matrix was probed by probes made from λ DNA (˜40 kb). It is clear that in this case more areas of the matrix show hybridization (FIG. 9B). To show that the DNA molecules from the 8 libraries are spread in the nylon membrane, the membrane was probed with radioactive labeled all possible 20-mers (4²⁰ different sequences). As shown in FIG. 9C, the radioactive 20-mer random sequences almost hybridized to the whole matrix space indicating that the matrix indeed contains duplex DNA molecules cover the whole space.

Fundamentally, an easily synthesized mixture of large number of DNA strands with length up to 100-mer and up to more than ˜10¹⁷ different sequences can be distributed on a two dimensional space. Different sequences end up at different locations in this space. The distribution of different sequences is systematic and continuous so that sequences with minimal differences end up next to each other. The matrix is universal, easy to make (low cost), and can be made with different ranges of distributions of oligo-probes for different applications. A collection of fixed short sequences can be added to the matrix to generate a grid inside the matrix for calibration.

The matrix can be used as a genome visualizer that depicts multi-genomic information as an image. Some parts of the image separate DNA sequences unique to each of the multiple genomes that may be present. More detail is robustly available by exploiting redundancy, signal processing, and underlying physical models of the matrix. It is important to note that the matrix is “universal” that is, it is not limited by biological assumptions. It can be used to obtain “signatures” of any input DNA or RNA samples.

The matrix is self calibrating, because it seeks to achieve recognizable global patterns rather than readout of a specific DNA sequences. For example, one can read out and store the image of each genome, instead of reading out the genomic DNA sequence and storing it. Signals from adjacent positions in a genome are mutually reinforcing and correlated. Thus, signal-processing tools can extract enhanced information.

In another aspect, the matrix genomic visualizer can also function as a purification tool. If portions of the readout are unique to some genome within a contaminated collection of DNA; the DNA hybridized on the matrix can be extracted from these physically separated regions. The extracted DNA can then be PCR amplified, and if desired, sequenced. This selectively amplifies the desired genome corresponding to the unique portions of the readout.

One of the major potential applications of the matrix is in the early detection of human disease by analyzing the “signatures” of a healthy individual and compared it to the pre-generated disease “signatures”. The genomic matrix system proposed here should be also capable of capturing a snapshot of an entire biosystem's state. At a later time, the snapshot can be updated, or a new one acquired, and compared to a previous memory to measure changes such as the appearance of pathogens. For example, this tool could be used in conjunction with target populations for in situ biomonitoring for applications such as broad-spectrum bioterrorism agents in water supplies. The goal is to use genomic information at the population or community scale to monitor and detect the existence of new biota (such as pathogens) in the environment. The scope of organisms with their genomic DNA sequenced is fairly small. Thus, much information at the genomic level is not available with conventional techniques. The genomic matrix provides a way to access information from all organisms in a community, to assess impact by human and non-human biomaterials, does not require explicit sequence knowledge, and is quick, flexible, and inexpensive to implement. Thus, it could provide a better diagnostic tool for ecological monitoring that provides a holistic view of the genomic status of an ecosystem.

Furthermore, snapshots acquired under different conditions can be compared to reason about common or similar mechanisms or effects, or merged to form a combined representation. Thus, it will be also possible to study the overall gene expression pattern changes without knowing exactly which genes are turned on or off as environmental conditions change to monitor the health of environments or to predict environmental impacts. That could apply not only to the external environment but also to the ones inside human body. For example, a physiological condition may be indicated by expression levels of many genes and complex relationships among the bacteria inside human body, and changes in those bacterial communities contributed to diseases. Therefore, it is possible to find patterns in the bacterial population that will predict when someone is about to get sick.

### Example 2

### Two-Dimensional Universal Oligonucleotide Matrix

The principle of the approach is to create a two-dimensional matrix with about 10¹⁸ oligonucleotides of 30- or 40-mers (FIGS. 2-7). In an exemplary model, the process is as follows (See FIG. 2): First a single-stranded oligonucleotides are synthesized—from about 30 to about 60-nucleotides long, containing a random sequence at its 5′ end and a 20-mer fixed sequence at the 3′ end that serves as the primer site to provide for conversion of the ssDNA library to a dsDNA library. The number of the different sequences in this library may be limited by the DNA synthesizer employed. For example, the current limitation is about 1 micromole which is about 6×10¹⁷ DNA molecules, thus being the upper limit of the different sequences from the two-dimensional matrix of the invention. However, it is expected that the technology will improve with time and greater oligo libraries will be possible.

After converting the approximately 10¹⁸ oligonucleotides (i.e., 30-60mer random polynucleotide segment and 20mer primer polynucleotide segment) ssDNAs to ds DNAs by simple primer extension reactions using DNA polymerase, the dsDNA library is subjected to a first dimension denaturing gradient gel electrophoresis (FIG. 3). On this first dimension gel electrophoresis, the denaturing strength inside this polyacrylamide gel changes continuously from 0% to 100% (7M urea plus 40% formamide) top to bottom, respectively.

Once electrophoresis is started, the whole population of the duplex DNA molecules begin to encounter some low degree of denaturing strength (from the urea and formamide). Those dsDNA molecules that have the lowest melting temperature begin to denature, while those having higher melting temperatures remain as duplex DNAs, and will denature later. The completely denatured duplex DNA molecules now comprise two single-stranded molecules, and, as such, run faster than the duplex DNAs under the denaturing conditions. As a consequence, the entire duplex DNA library can be separated according to their denaturing temperatures (i.e. the hydrogen bonding strengths between the two partner single strands in a duplex molecule). As shown in FIGS. 4-6.

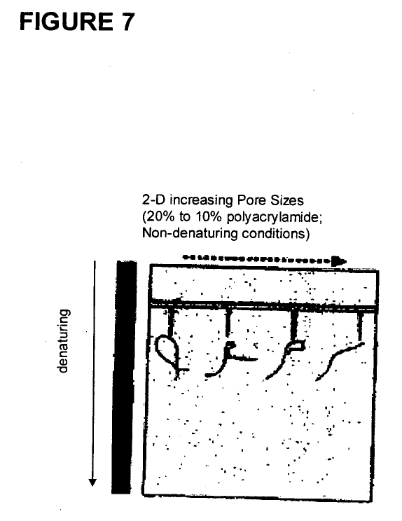

Thereafter, the strip of the first dimension gel with the denatured DNA is cut out (FIG. 7) and placed on the top of a second gel. As shown in FIG. 7, the DNAs in the first gel (which are now separated according to their melting temperatures) all have roughly the same melting temperature and have migrated to about the same position in the first dimension of electrophoresis (as indicated by the box). The second gel, on the other hand, is preferably a native gel. Instead of denaturing the DNA, the second gel functions to maintain the secondary structures of the ssDNAs that have about the same melting temperature. In this second, native gel, separation of those ssDNAs is accomplished according to their secondary structures formed by the intra-molecular hydrogen bonds. The DNA molecules further separated in the second gel result in a smear on the two-dimensional space on the second gel.

The second gel is then used to make a probe, such as by blotting it onto a nitrocellulose membrane using capillary transfer or electrophoresis. The DNA is fixed to the hybridization support, for example, a nylon membrane by exposure to short wave UV light at an intensity of 5000 Joules/cm2 in a Stratalinker (Stratagene Cloning Systems). The membrane can then be used as the universal probe for detection of any large input DNA or RNA labeled with fluorescent or radioactive labels. The resulting fluorescent or radioactive signals of the input DNA or RNA can then be analyzed to generate a “signature” or the specific input DNA or RNA (FIG. 3). (See, Southern, E. M. 1975. J. Mol. Biol. 98:503 Sambrook, J., Fritsch, E. F., and Maniatis, T. 1989. Molecular Cloning, A Laboratory Manual, 2nd Ed. Cold Spring Harbor Laboratory Press, Cold Spring Harbor, N.Y.).

Positive hybridization signals are detected by machinery such as, in the case of fluorescently labeled probes, a confocal microscope that scans the array and detects the presence of labeled nucleic acid molecule (DeRisi, et al., Nature Genetics 14:457-460; Lockhart, et al., Nature Biotechnology 14:1675-1680). Scanning and detection systems using focused laser beams are available from Affymetrix (GeneArray™ Scanner, Santa Clara, Calif., http://www.affymetrix.com), General Scanning (ScanArray™ Scanner, Menlo Park, Calif., http://www.genscan.com), and Incyte (GemArray Scanner, Palo Alto, Calif., http://www.incyte.com), among other companies. If the probes are radiolabeled, the array may be subjected to autoradiography or phosphorimaging (Chalifour, et al. Anal. Biochem. 216:299-304, Pietu, et al., Genome Research 6:492-503) on, for example, a Storm 840 Phosphor Imaging System (Molecular Dynamics); analyzed by ImageQuaNT analysis software to quantitate bands on images generated with the Storm 840 System. Other methods of detection may be used in accordance with the nucleic acid molecule labeling techniques that may be used in accordance with the nucleic acid molecule labeling techniques that may be used. The position of the labeled nucleic acid molecule may be localized on the array to identify the specific nucleic acid molecules, such as DNA molecules, on the array that have hybridized to the isolated nucleic acid molecule fragment (Lockhart, et al., Nature Biotechnology 14:1675-1680; DeRisi et al., Nature Genetics 14:457-460: Chalifour, et al. Anal. Biochem 216:299-304 and Pietu, et al., Genome Research 6:492-503). Software is commercially available to facilitate the localization and determine the intensity of positive hybridization signals (the GeneChip Workstation Expression Data Mining Tool from Affymetrix, Santa Clara, Calif., the ScanArray™ Acquisition QukzantArray™ Tools from General Scanning, Menlo Park, Calif., and the GemTools™ LifeArray™ system from Incyte, Palo Alto, Calif.). Positively hybridizing nucleic acid molecules, such as DNA molecules, whether of known or unknown identity, are derived from genes expressed by the cell, tissue, or organ of the species used.

While embodiments and examples of the invention have been shown and described herein, it will be understood that such embodiments are provided by way of example only. Numerous variations, changes and substitutions will occur to those skilled in the art without departing from the spirit of the invention. Accordingly, it is intended that the appended claims cover all such variations as fall within the spirit and scope of the invention.

What is claimed:

1. An oligonucleotide for performing nucleic acid analysis comprising a random polynucleotide segment of from about 10 to about 60 nucleotides in length linked to at least one known primer polynucleotide segment of from about 10 to about 50 nucleotides in length.

2. The oligonucleotide of claim 1, wherein the random polynucleotide segment is from about 25 to about 50 nucleotides long.

3. The oligonucleotide of claim 1, wherein the primer polynucleotide segment is from about 15 to about 25 nucleotides long.

4. The oligonucleotide of claim 1, wherein the random polynucleotide segment and the primer polynucleotide segment comprise RNA nucleotides, DNA nucleotides, PNA nucleotides or combinations thereof.

5. An oligonucleotide matrix comprising
an oligonucleotide library comprising about 4ⁿ individual oligonucleotides, wherein n represents the number of nucleotides in a random polynucleotide segment and is an integer from 10 to 60, and wherein the random polynucleotide segment is covalently linked to at least one known primer polynucleotide segment having from about 10 to about 50 nucleotides; and
a hybridization support, wherein the individual oligonucleotides are distributed on the hybridization support as determined by their melting temperature in a denaturing gel.

6. The oligonucleotide matrix of claim 5, wherein the random polynucleotide segments are from about 25 to about 50 nucleotides long.

7. The oligonucleotide matrix of claim 5, wherein the primer polynucleotide segments are from about 15 to about 25 nucleotides long.

8. The oligonucleotide matrix of claim 5, wherein the primer polynucleotide segment comprises at least one feature selected from the group consisting of a restriction endonuclease consensus sequence, a methyl group, a peptide group, a fluorescent label, a radioactive label, at least one modified nucleotide or a combination thereof.

9. A method for generating an oligonucleotide set for array analysis comprising the steps of:
synthesizing a plurality of random polynucleotide segments of length n by performing an oligonucleotide synthesis reaction in the presence of equal molar concentrations of nucleotides A, C, T (or U), and G or derivatives thereof, wherein n represents the number of nucleotides in the random probe segment and is an integer from 15 to 50;
linking said random segments to at least one primer polynucleotide segment of from about 10 to about 40 nucleotides in length to generate, and wherein the number of oligonucleotides in the set is approximated by the formula, 4ⁿ; and optionally performing polymerase chain reaction (PCR) under suitable conditions to amplify the oligonucleotides.

10. A method for generating an oligonucleotide matrix comprising the steps of:
generating at least one oligonucleotide set according to the method of claim 9;
performing denaturing gel electrophoresis separation of the oligonucleotide set in a first dimension under suitable buffering conditions;
transferring the oligonucleotides in the to a hybridization support under suitable buffering conditions.

11. The method of claim 10, wherein the method further includes the step of performing a second electrophoresis separation in a direction perpendicular to the first prior to the step of transferring the oligonucleotides to a hybridization support, wherein the second electrophoresis separation is performed on a non-denaturing gel under suitable buffering conditions.

12. The method of claim 11, wherein the second dimension lies within the same plane and is separated by 90 degrees from the first dimension.

13. The method of claim 10, wherein the denaturing gel comprises a gradient of from about 0% to 100% of a mixture comprising from about 3M to about 9M urea, and from about 20% to about 60% formamide.

14. The method of claim 13, wherein the non-denaturing gel comprises a polyacrylamide gradient of from about 20% to about 10%.

15. The method of claim 10, wherein the hybridization support comprises a member selected from the group consisting of nitrocellulose, glass, DEAE dextran, PVDF, nylon, silicon, and combinations thereof.

16. A method for performing nucleic acid analysis comprising:
providing the oligonucleotide matrix of claim 10;
providing an input nucleic acid from a biological or synthetic source, wherein the input nucleic acid comprises a suitable detection label;
incubating the input nucleic acid with the oligonucleotide matrix under suitable hybridization conditions; and
detecting the labeled input nucleic acid bound to the oligonucleotide matrix.

17. The method of claim 16, wherein the input nucleic acid comprises RNA, DNA, nucleotide analogs, or combination thereof.

18. The method of claim 16, wherein the label comprises a fluorophore, a radioactive isotope, a peptide, a modified nucleotide or a combination thereof.

19. A method of claim 16, further comprising the steps of:
using the method of claim 16 to generate a reference signature database based on input nucleic acid from a known biological source,
generating a test signature from nucleic acid of interest using the matrix of the invention; and
comparing said test signature to the reference signature database.

20. The method of claim 19, wherein the reference signature database comprises at least one signature of a biological source selected from the group consisting of a virus, bacteria, fungus, yeast, alga, protist, plant, animal, and any combination thereof.

21. A method for monitoring or diagnosing diseases, disease progression, and/or disease states in a subject comprising:
using the method of claim 16 to generate a diagnostic signature database based on input nucleic acid from a normal or healthy biological source and said biological source in an abnormal or diseased state;
generating a test signature from nucleic acid of another biological source whose pathological state is unknown using the matrix of the invention; and
comparing the test signature to the database of diagnostic signatures to diagnose the particular disease or disease state.

22. A method for monitoring the efficacy or response of therapeutic dose upon a subject comprising:
using the method of claim 16 to generate a diagnostic signature database based on input nucleic acid from a first subject during successful treatment with a therapeutic;
generating a test signature from nucleic acid of a second subject in response to known levels of said therapeutic using the matrix of the invention; and
comparing the test signature to the database of diagnostic signatures to determine proper treatment regimen for said second subject.

23. A method of identifying a therapeutic molecule comprising:
using the method of claim 16 to generate a drug class signature database based on input nucleic acid from at least one subject treated with a drug of known utility;
generating a test signature from nucleic acid of a second subject in response to treatment with a therapeutic agent of unidentified utility using the matrix of the invention; and
comparing the test signature to the drug class signature database.

24. A database comprising a collection of signatures generated according to the methods of claim 16.


SEQUENCE LISTING (24 sequences)

SEQ ID NO: 1 - DNA, 40 bp, Artificial
  synthetic oligo
atttttataaatgttagaaatttattattatctttaatat

SEQ ID NO: 2 - DNA, 40 bp, Artificial
  synthetic oligo
tcatatgtcatatttattattgtgtatttataaacatgat

SEQ ID NO: 3 - DNA, 40 bp, Artificial
  synthetic oligo
cttaacacgttatatttggcgtatgtttttatatgatcaa

SEQ ID NO: 4 - DNA, 40 bp, Artificial
  synthetic oligo
catctataccgttaatgcccaatacaataggacaattttt

SEQ ID NO: 5 - DNA, 40 bp, Artificial
  synthetic oligo
cattgatacccagttggttctaataatcatactgaacgtc

SEQ ID NO: 6 - DNA, 40 bp, Artificial
  synthetic oligo
ctcgtgtagtgagatttgcgtgctctattcgtttcgaagg

SEQ ID NO: 7 - DNA, 40 bp, Artificial
  synthetic oligo
ctgggtaaagcagcgttcaagtggcaagtgatccgacctc

SEQ ID NO: 8 - DNA, 40 bp, Artificial
  synthetic oligo
ctttaggcccgaagtagggtggcccagtagacctaccggc

SEQ ID NO: 9 - DNA, 40 bp, Artificial; identical to SEQ ID NO: 1
  synthetic oligo

SEQ ID NO: 10 - DNA, 40 bp, Artificial
  synthetic oligo
ttattcaaaatattttctattatatttaaacatataaatt

SEQ ID NO: 11 - DNA, 40 bp, Artificial; identical to SEQ ID NO: 2
  synthetic oligo

SEQ ID NO: 12 - DNA, 40 bp, Artificial
  synthetic oligo
gataattttgccagagaaataatattcaaataaaatgaat

SEQ ID NO: 13 - DNA, 40 bp, Artificial; identical to SEQ ID NO: 3
  synthetic oligo

SEQ ID NO: 14 - DNA, 40 bp, Artificial; identical to SEQ ID NO: 4
  synthetic oligo

SEQ ID NO: 15 - DNA, 40 bp, Artificial
  synthetic oligo
tttaacatgattcttgtgtgggttgtgtatatcgcatcta

SEQ ID NO: 16 - DNA, 40 bp, Artificial; identical to SEQ ID NO: 5
  synthetic oligo

SEQ ID NO: 17 - DNA, 40 bp, Artificial
  synthetic oligo
agtaggtgagatagaggagaactcagactcatggtaagct

SEQ ID NO: 18 - DNA, 40 bp, Artificial
  synthetic oligo
tgcattcaatctcgtgtcaatggagaagacacgactggtg

SEQ ID NO: 19 - DNA, 40 bp, Artificial; identical to SEQ ID NO: 6
  synthetic oligo

SEQ ID NO: 20 - DNA, 40 bp, Artificial
  synthetic oligo
ctcaggatcttcatgctacgcccagccgatgtacggttga

SEQ ID NO: 21 - DNA, 40 bp, Artificial; identical to SEQ ID NO: 7
  synthetic oligo

SEQ ID NO: 22 - DNA, 40 bp, Artificial; identical to SEQ ID NO: 8
  synthetic oligo

SEQ ID NO: 23 - DNA, 40 bp, Artificial
  synthetic oligo
ggcccggctgcgggtacttgaacctgactcttgccgggcc

SEQ ID NO: 24 - DNA, 40 bp, Artificial
  template synthetic oligo
actataacaaaaccgctgagcacacactgtgacgtggccg